(19) United States
(12) Patent Application Publication
(10) Pub. No.: US 2013/0053446 A1
(43) Pub. Date: Feb. 28, 2013

(54) PHARMACEUTICAL PREPARATIONS HAVING INDIVIDUALIZED DOSAGE AND STRUCTURE

(71) Applicants: Fernando J. Muzzio, Sparta, NJ (US); Pavlo Takhistov, East Brunswick, NJ (US); Bozena B. Michniak-Kohn, Piscataway, NJ (US)

(72) Inventors: Fernando J. Muzzio, Sparta, NJ (US); Pavlo Takhistov, East Brunswick, NJ (US); Bozena B. Michniak-Kohn, Piscataway, NJ (US)

(73) Assignee: Rutgers, The State University of New Jersey, New Brunswick, NJ (US)

(21) Appl. No.: 13/508,485

(22) PCT Filed: Nov. 6, 2010
(86) PCT No.: PCT/US10/55752

§ 371 Date: Oct. 31, 2012

Related U.S. Application Data
(60) Provisional application No. 61/258,670, filed on Nov. 6, 2009.

(51) Int. Cl. B67D 7/06; A61K 31/137; A61K 31/192; A61P 35/00; A61P 25/00; A61P 37/08; A61P 31/18; A61P 5/00; A61P 7/02; A61P 1/00; A61P 1/16; A61P 1/06; A61P 21/02; A61P 7/10; A61P 15/00; A61P 25/22; A61P 25/06; A61P 23/00; A61P 23/02; A61P 25/20; A61P 25/08; A61P 29/00; A61P 25/26; A61P 37/00; A61P 33/00; A61P 31/00; A61P 3/02; A61K 31/18

(52) U.S. Cl. 514/570; 514/592; 514/654; 222/23

(57) ABSTRACT

A manufacturing device capable of creating individualized dosages of pharmaceutical preparations in which a metering system and a means of performing non-destructive chemical analysis of individual manufactured units are controlled by a microprocessor to precisely control the content and structure of each individual unit.

### CROSS-REFERENCE TO RELATED APPLICATIONS

This application claims priority under 35 U.S.C. §119(e) to U.S. Provisional Application Ser. No. 61/258,670, which was filed on Nov. 6, 2009. The disclosure of this application is incorporated herein by reference.

### BACKGROUND OF THE INVENTION

Drug products are currently developed, approved and manufactured using a regulatory process where companies develop a fixed formulation and a batch manufacturing method for making large numbers of unit doses that meet certain quality specifications, typically determined by narrow ranges of drug content, dissolution/bioavailability, etc. Each variation of the formula is applied for and approved individually. Each one is manufactured separately, typically in “batches” ranging from 100,000 to a few million product units.

This approach to product development and manufacturing has a number of adverse consequences. First, companies typically need to maintain large and expensive stocks of finished products for each of the approved potencies. Second, because these products are manufactured in large numbers of units, they need to have long shelf lives.

Demonstrating stability over shelf life is both a time consuming process that substantially delays introduction of new products to market, and is inherently risky, because it is initially based on a surrogate measure where products are stressed by cycling them through temperature and moisture ranges.

Because the manufacturing method is geared towards a small number of approved versions of the finished products, late phase clinical trials avoid testing formulation variations other than those for approved doses. In fact, at the present time, effective technology for making clinical supplies for many variations of a given product is unavailable.

A consequence of the large batch approach is that the manufacturing process, once approved by the FDA, is expected to remain unchanged, and to be repeated for every batch ever manufactured. An undesirable by-product of this approach has been the extremely slow technological evolution of pharmaceutical products and processes.

It is envisioned that, in the near future, health care will be focused on the optimization of medication therapy and the provision of patient-centered and population-based care. Central to this vision is the concept of personalized medicine. In short, personalized medicine requires the matching of the patient and diagnosis with the right drug (or combination of drugs) and the right dose given at the right time.

With over 4 billion prescriptions dispensed annually in the United States, the inability to manufacture and distribute individualized dose forms to patients is currently and will remain a major roadblock to the realization of personalized medicine. Fortunately, pharmacy benefit managers, like Medco, have pushed for advances in informatics and robotics allowing for high-throughput prescription processing and distribution. However, the realization that current pharmaceutical manufacturing plat-forms are wholly unable to supply individually formulated products under approvable conditions has not been widely recognized. As such, even if we knew how to determine the optimum dose and dose regimen for millions of individual patients, the existing manufacturing and regulatory infrastructure is incapable of manufacturing the billions of personalized prescriptions they will require.

Therefore, it would be desirable to provide a mechanism for the manufacture of medicines having individualized dosing and regimen regimes to be able to achieve precisely controlled in vivo performance.

Under such a regime, neither pharmaceutical companies nor the commercialization system will need to maintain large stocks of finished products. Instead, they will be able to stock the raw materials in very much the same way that cartridges containing different colors of ink for printers are stocked. This will generate very large savings to drug manufacturers; savings that should at least in part be passed to the patient population.

Because products will be manufactured as needed by individuals, most products will require a shelf life of a few weeks at most. For long-term therapies and chronic condition treatments, the same system can be used to automatically generate a fresh supply of medication at periodic intervals. The entire set of delays associated with demonstrating physical and chemical stability before product approval will be reduced to (1) assuring product stability for a few weeks (a situation that can be tested “for real,” rather than using the “accelerated stability under stress” surrogate) and (2) assuring the stability of raw materials, which is much more easily accomplished than assuring the stability of the finished product. Moreover, eliminating the need to assure long-term product stability for every batch manufactured, and the dramatic changes in quality testing that will be necessarily implemented, will also accrue enormous savings that should result in lower cost of medicines.

### SUMMARY OF THE INVENTION

The present invention addresses these needs. It has now been discovered that a microprocessor can be interfaced with a metering system to provide for the precise deposition of micro-quantities of one or more pharmaceutically active agents and a chemical analyzer to non-destructively verify the composition and structure of said deposition to control precisely the manufacturing of a custom dose and a plurality of custom doses of one or more pharmaceutically active agents.

Therefore, according to one aspect of the present invention, a system for the manufacture of a custom dose of one or more pharmaceutically active agents is provided, which combines:
a. a metering system, to provide for the precise deposition of micro-quantities of one or more pharmaceutically active agents;
b. a chemical analyzer, to non-destructively verify the composition and structure of said deposition; and
c. a microprocessor interfaced to the metering system and the chemical analyzer to control the deposition and verification of said pharmaceutically active agent.

According to one embodiment, the system may be configured to deposit more than one pharmaceutically active agent into a single pharmaceutically acceptable medium. According to another embodiment, the system may be configured to deposit a different concentration of each of the pharmaceutically active agents. According to yet another embodiment, the system is configured to manufacture a plurality of different custom doses. In another embodiment, the system is configured to manufacture a plurality of the same custom doses.

In another embodiment, the system is configured to also provide one or more pharmaceutically acceptable excipients. The one or more pharmaceutically acceptable excipients are selected from surfactants, preservatives, stabilizers, biocompatible polymers, solvents, viscosity modifiers, absorption enhancers, mucoadhesives, solvents, buffers, acidulants, diluents, emulsifying agents, suspending agents, wetting agents, anti-caking agents, plasticizers, coating agents, sweetening agents, flavor enhancers, flavoring agents, coloring agents, adsorbents and antioxidants.

In another embodiment, the system further includes a mechanical handling system to handle a pharmaceutically acceptable medium into which the pharmaceutically active agents are deposited. In one embodiment, the medium is a non-consumable medium such as a syringe or a vial. In another embodiment, the medium is a consumable medium such as a capsule.

In another embodiment, the pharmaceutically active agent is selected from chemotherapeutic agents, agents for treating central nervous system disorders, agents for treating allergic reactions, agents for treating attention deficit disorder, micro-nutrients, vitamins, agents for treating human immunodeficiency virus, hormone therapy agents, anticoagulants, highly potent bio-pharmaceuticals, agents for treating pediatric disorders, agents for treating geriatric disorders, diagnostic agents, radio-pharmaceutical agents, gastrointestinal drugs, liver drugs, blood, fluids, electrolytes, hematological drugs, cardiovascular drugs, respiratory drugs, sympathomimetic drugs, cholinomimetic drugs, adrenergic antagonists, adrenergic neuron blocking drugs, antimuscarinic drugs, antispasmodic drugs, skeletal muscle relaxants, diuretic drugs, uterine drugs, anti-migraine drugs, hormones, hormone antagonists, general anesthetics, local anesthetics, anti-anxiety drugs, hypnotic drugs, antiepileptic drugs, psychopharmacologic drugs, analgesics, antipyretics, anti-inflammatory drugs, histamine, anti-histaminic drugs, central nervous system stimulants, anti-neoplastic drugs, immunoactive drugs, parasiticides, immunizing agents, allergenic extracts, anti-infectives, enzymes, nutrients, vitamins, micronutrients, nutraceuticals and pesticides.

In one embodiment, the system microprocessor includes memory programmed with a database characterized by information on various pharmaceutical preparations and with instructions for controlling the metering system and the chemical analyzer of the system to create various pharmaceutical preparations. In another embodiment, the system includes a reading or communication device for receiving a dosage formulation from a paper or electronic prescription.

In one embodiment, the system microprocessor is programmed for the preparation of geriatric doses. In another embodiment, the system microprocessor is programmed for the preparation of pediatric doses.

The present invention also provides methods by which custom doses of at least one pharmaceutically active agent are prepared utilizing the system of the present invention. Methods according to the present invention include the steps of providing instructions to the microprocessor selecting the custom dose to be prepared and commanding the microprocessor to operate said system to prepare the custom dose.

In one embodiment, the instructions select a plurality of different custom doses. In another embodiment, the instructions select a plurality of the same custom doses.

Custom pharmaceutical doses are also provided, containing at least one pharmaceutically active agent, which are prepared according to the methods of the present invention. According to one embodiment, the doses contain more than one pharmaceutically active agent in a single pharmaceutically acceptable medium. Doses according to this embodiment may contain different concentrations of each of the pharmaceutically active agents.

A plurality of the doses are also provided characterized by either a plurality of different custom doses or a plurality of the same custom doses. The doses may be formulated for a geriatric patient or for a pediatric patient. The dose may be based on a consumable or a non-consumable pharmaceutically acceptable medium. Examples of pharmaceutically acceptable media include capsules, syringes and vials.

According to one embodiment of a method according to the present invention, the dose is prepared prior to dispensing to a patient. In another embodiment the dose is prepared prior to dispensing to a participant in a clinical trial. In either embodiment, the dose may be formulated for a pediatric patient or for a geriatric patient.

Methods according to the present invention also prepare the dose prior to dispensing to a participant in a clinical trial or in the course of pharmaceutical research. The clinical trial or pharmaceutical research may require a formulation for a pediatric or geriatric patient.

The present invention also includes methods in which the dose is prepared to order from an order or prescription issued by a professional with authority to prescribe or order the dispensing of drugs. In addition to the familiar dispensing of drugs by a pharmacist at a retail pharmacy from a prescription issued by a physician or nurse practitioner, embodiments according to this method include the dispensing of doses by a pharmacist at a hospital pharmacy, and by authorized personnel in battle-fields, quarantine zones and other isolated areas.

### BRIEF DESCRIPTION OF THE DRAWINGS

FIG. 1 is a schematic representation of one embodiment of a drop-on-demand micro-dispensing system;

FIG. 2 is a graph showing the correlation between the opening time of the valve and the fluid mass dispensed after 100 cycles;

FIG. 3 is a flow chart of the software system for generating the control pulse;

FIG. 4 is an acquired time history of the voltage signal generated by the system of FIG. 3, used to dispense droplets of various masses;

FIG. 5 is a flow chart of the software system for performing weight measurement and verification;

FIG. 6 is a flow chart of the software system for controlling movement of the motorized stage for the handling of the media on which the materials will be dispensed;

FIG. 7 is a graph showing the dynamic response of the weighting module for single droplets generated at various valve opening times;

FIG. 8 is a graph showing the dynamic response of the weighting module for single droplets generated for various gas pressure values;

FIG. 9 is a graph showing the mass of model fluid dispensed systematically (on a tray) as a function of valve opening times; and

FIG. 10 is a graph showing the relative standard deviation of the dosage mass (dosages were 100 aliquots generated at different valve opening times)

### DETAILED DESCRIPTION OF THE INVENTION

Disclosed herein are systems and methodologies for the manufacture of personalized product doses at the commercial scale. The goal is the ability to economically manufacture small numbers of product units with pre-determined composition, structure and drug release profiles that can be formed, analyzed and approved for release with high quality and consistency and a minimum of human intervention.

To achieve the objects of the invention, highly accurate metering systems can be used to deliver multiple drugs and other ingredients to commonly known drug delivery media, such as, for example, capsules (hard or soft), syringes, vials (glass or plastic), cellulose strips, and hard tablets. Such metering systems include, but are not limited to, electrospraying, electropulsing, acoustic pulsing, actuated pressure waves (“drop-on-demand”), microneedles and several other methods capable of metering droplets of solutions and nano/micro suspensions into small cavities.

In preferred embodiments of the invention drop-on-demand (DoD) technology is used for creating reliable, approvable platforms suitable for the manufacture of personalized therapeutics. Drop-on-demand methods, commonly used in ink-jet printers, use acoustic and/or electrical fields to create and target fluid drops with extremely accurate control of size and composition. With this technology, nano-liter level control is achievable. Drops generated using this technology may consist of solutions, solid/liquid and liquid/liquid suspensions, or melts, which can be used as building blocks for creating complex structures with an extremely high precision and versatility. Applied to precision dosage formulations, a drop-on-demand system can be used to drop precise dosages of formulated drug compounds onto edible, (e.g. biopolymeric) substrates creating functional and convenient drug delivery systems.

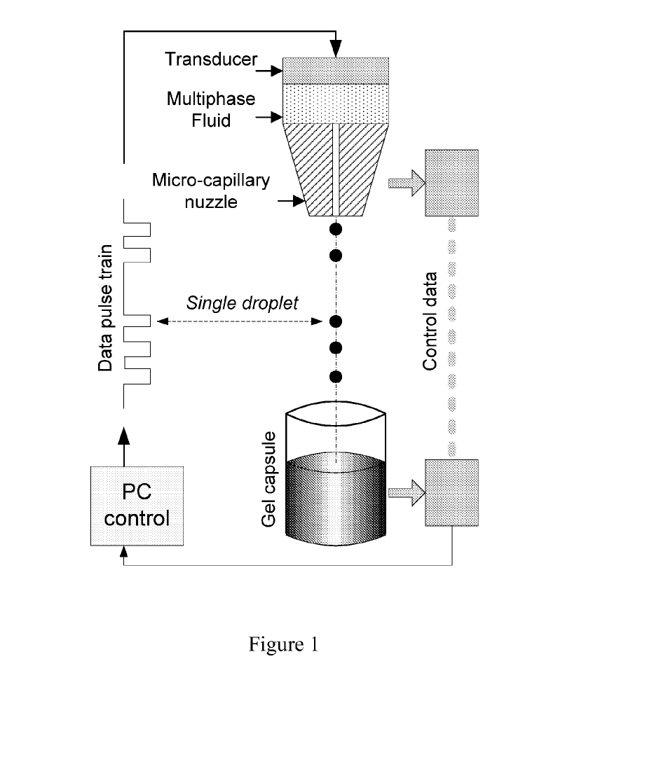

There is a broad range of diverse technologies that fall into the DoD category. The physics and the methods employed within this group may differ substantially, but the end result is consistent generation of small droplets of fluid. Most of these methods fall into two general categories, continuous mode and demand mode. Generally, in DoD dispensing devices, the fluid is maintained at certain pressure and a transducer is used to create a drop only when needed. The transducer creates a volumetric change in the fluid which creates pressure waves. The pressure waves travel to the orifice and are converted to fluid velocity, which results in a drop being ejected from the orifice. Drops generated using these methods can be solutions, solid/liquid and liquid/liquid suspensions, or melts, which can be used as building blocks for creating complex structures with an extremely high precision and versatility. FIG. 1 shows a drop-on-demand ink-jet system in schematic form.

The use of inkjet dispensing technology generally provides several advantages over traditional syringe-pump based liquid handling methods. As a non-contact process, the volumetric accuracy of ink-jet dispensing is not affected by how the fluid wets a substrate, as is the case when positive displacement or pin transfer systems “touch off” the fluid onto a substrate during the dispensing event. In addition, the fluid source cannot be contaminated by the substrate, as is the potential during pin transfer touching. Because the technique is non-contact delivery of the drop to a small location is not limited by the mechanical size of the tip. The ability to free fly the droplets of fluid over a millimeter or more allows fluids to be dispensed into wells, hollows or other substrate features (e.g., features that are created to control wetting and spreading). Finally, the speed of inkjet printing translates into high-speed dispensing when using these technologies.

Inkjet printing is a non-impact/contact printing technology in which drops of tens of picoliters are jetted from a small capillary orifice (usually less than 100-μm in diameter) onto a designated position on a substrate, such as inkjet paper. As indicated by its name, DoD inkjet printing involves devices that eject drops only when demanded. Demand mode ink-jet systems have no fluid recirculation requirement, and this makes their use as a general fluid microdispensing technology more straightforward than continuous mode technology.

The basic principle of inkjet micro-dispensing involves some means of compressing the liquid against a small orifice to create sufficient linear force to eject the fluid in the form of a drop. Inkjet technologies differ by the means used for creating the compressive force. DoD can be subdivided by distinguishing the mechanism by which a drop is ejected, namely piezoelectric, solenoid and positive displacement type.

In a piezoelectric drop ejection system, electric pulses are applied to a piezoelectric material and mechanical motions are produced which induce pressure waves in a capillary tube, causing ink to be squeezed out of a nozzle opening.

In a solenoid drop ejection system, dispensing force is built when pneumatic or hydraulic pressure is used to compresses fluid against a valve. When the valve is actuated using electric discharge a drop is ejected.

In a physical displacement drop ejection system, a physical moving force is used to drive material out of the valve. Typically, either a screw or a rod/piston configuration is used to force the materials and eject a drop.

The application of inkjet technology to the delivery of functional materials poses a range of important challenges in terms of ink formulation, print head and print system design, substrate choice and preparation, and control of solvent evaporation. The inks need to be formulated in a narrow viscosity range compatible with the specific print head used. In many cases, the additives that are routinely used in graphic arts printing to modify, for example, ink viscosity, cannot be used for functional materials, as they will adversely affect the materials' performance. It is necessary to ensure that the ink does not in any way chemically interact with or dissolve any of the components inside the print head or the ink feed system. Nor should the ink's properties degrade under the high mechanical shear of a piezoelectric head or the high-temperature conditions of a thermal inkjet head. The ejection of droplets from the array of nozzles needs to be stable and reliable. Nozzles can become clogged by the evaporation of ink on the nozzle plate or the presence of particulates in the ink. Fluctuations in droplet volume can lead to undesirable variations in the amount of material deposited onto the substrate.

Preferably, such metering systems can be integrated into bench-top microencapsulator, syringe-filling and/or vial-filling machines. The system will also require integration of a non-destructive means of chemical analysis such that each sample of a given formulation may be tested for accuracy of composition.

Existing printing technology can be used to create multilayer structures on edible flat substrates, such as rice paper or cellulose substrates similar to those currently used for formulating Lysterine, Benadryl and other strip-film products. Single or multiple drugs, in solution or in nanosuspension form, can be deposited with extremely high precision onto flat substrates, which can be pre-coated or post-coated with biocompatible polymers to provide desirable release functionality and flavor. To implement this embodiment, existing printing technology may be modified to be able to formulate print-outs to achieve desired drug release profiles for individual patients.

Additionally, individualized doses of pharmaceuticals can be “printed” on the surface of placebo tablets, or in a hollow carve-out on the surface of placebo tablets. Existing drop-on-demand mechanical systems may be modified to allow the creation of multilayer structures on the surface of placebo tablets.

In the present invention, drop-on-demand methods can be used for filling hard gelatin capsules with high precision. In a variation of this embodiment, the deposited drops may be allowed solidify into particles by, for example, evaporating a solvent or by solidifying a molten polymer, before allowing the drops to enter the capsule. As such, multiple drugs can be dosed to high precision within a single capsule. To implement this embodiment, existing small scale encapsulation equipment may be adapted to include the drop-on-demand capabilities and the non-destructive chemical analysis capabilities. Drop-on-demand methods can be used similarly to fill syringes and vials with high precision.

To achieve the desired in vivo performance for individual patients it is necessary to assure the microstructural parameters of the product units be controlled and verified. Currently, pharmaceutical product quality is assured by extracting and testing samples from each manufactured batch using destructive methods.

In the present invention, because the products are manufactured in the precise amounts needed by individual patients, quality and performance must be examined and assured for each product unit at the point and time of manufacture. Such verification can be achieved using any number of currently known chemical analysis methods including, but not limited to, near infra-red, Raman, confocal scanning microscopy and X-rays. One or more of these technologies can be integrated into small product forming devices to assay non-destructively every product unit, in much the same way that every computer chip is tested in microelectronics manufacturing, to assure that it contains the appropriate composition and structure.

The flexible dose manufacturing method of the present invention is also suitable for use in other applications unrelated to personalized medicine, such as dispensing in hospitals during acute care and product development, both of which requires making small number of product units under variable composition. The method can also be used to manufacture supplies for clinical studies, where single product/dosage units of many different potencies of multiple drug combinations are needed.

This approach will profoundly change pharmaceutical manufacturing, not only in the process itself, but also in availability of drugs. Currently, companies deal with their own drugs or (for generic companies) specialize in certain groups of compounds. Flexible dose manufacturers will utilize a wide array of drugs in pure form from multiple manufacturers or other “raw material” form (solution, nanosuspension, etc.) that may be used, for example, in the drop-on-demand platform for forming hundreds or thousands of different product variations.

The present invention will trigger a profound change in how pharmaceutical products are developed, approved and manufactured. The system enables personalized medicine in two critical ways: (1) by making it possible to create product units of precisely controlled composition and structure, which can then be used to fine-tune in vivo performance, and (2) by creating a manufacturing technology platform suitable for personalized dosing once knowledge is available.

The present invention also has other benefits which will profoundly alter the existing drug development paradigm. First, it will facilitate product development by enabling the pharmaceutical scientist to change the product composition quickly under conditions virtually identical in quality and precision to those to be used at a later stage during commercial manufacturing. Second, it will also facilitate the manufacture of clinical supplies with finely controlled composition and structure, making it possible to study much more precisely how patients respond to complex treatment options. Third, it will provide generic platforms for formulating a wide diversity of materials with precisely controlled in vivo performance, simplifying (and lowering the cost of) the drug development and approval process.

In implementation, these technologies will be integrated into small, automated machines that will create the product unit, analyze it and approve it for release. Such machines may be controlled by computer microprocessor and may have a database of drug formulations and instructions for manufacture of personalized dosages. Under computer or microprocessor control, the machine may be directed, based on the data-base content, to create precisely-controlled individualized dosages of pharmaceuticals.

Because the manufacture of each dose for each patient will be computer-controlled, the proposed technologies will provide a natural framework for collecting data regarding products administered to each patient, minimizing risk of error, allowing better monitoring of prescription interaction, and minimizing prescription-sharing among patients. Over time, such information will become an enormous data-mining resource, which will enable much more detailed epidemiological studies and will promote scientific learning beyond our current ability to anticipate.

The following non-limiting examples set forth herein below illustrate certain aspects of the invention.

### EXAMPLES

### Materials

Drugs were dissolved into two different solutions. The first solution was an ethanol:water mixture at a ratio of 7:3 (by weight). The second solution was also an ethanol to water mixture of 7:3 but PEG 400 was added at a ratio of 1:24 (ratio of PEG to ethanol-water solution).

Chlorpropamide drug solution was formed by dissolving 0.40 grams of chlorpropamide into a 7:3 solution mixture of ethanol:water. Dopamine hydrochloride and ibuprofen solutions were formed by individually dissolving 0.40 grams of drug into an ethanol:water mixture of 7:3. Additional drug solutions of chlorpropamide were formed by dissolving 0.40 grams of drug into the ethanol-water-PEG solution. A dopamine hydrochloride solution of DI water was formed at a ratio of 1:45 by wt. %.

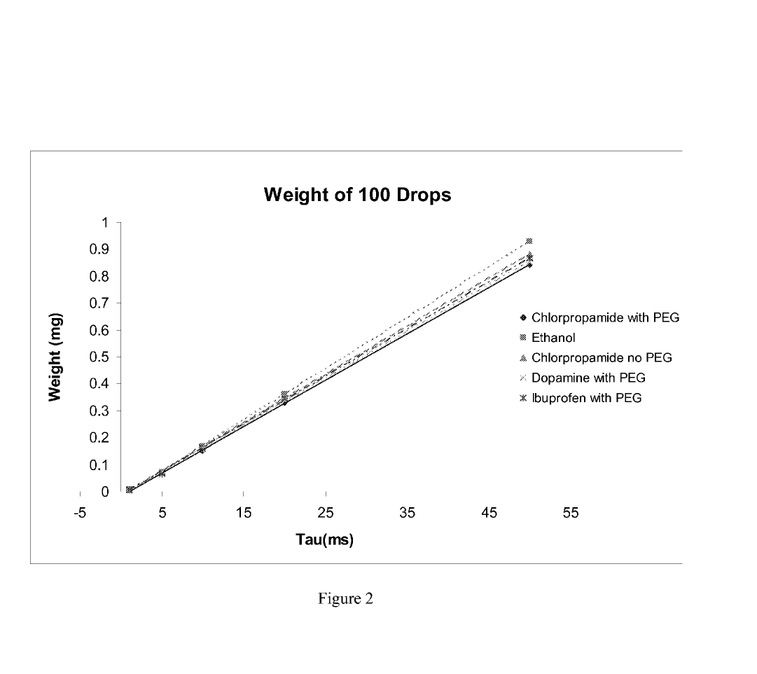

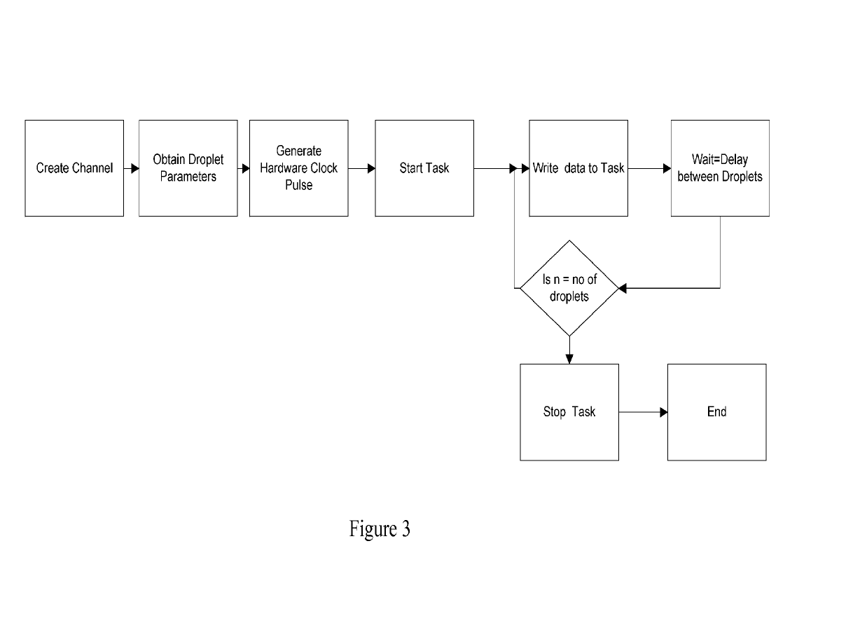

The linear correlation between the opening time for the valve and the mass of solution dispensed after 100 cycles can be seen in the FIG. 2. However, different amounts of the different fluids pass through the valve while it is open for a specific period of time. A correlation can be seen between the viscosity and the mass of fluid dispensed. The mass of liquid dispensed increases from ‘CP with PEG’, a comparably viscous solution, to ethanol which is least viscous. However, the correlation between valve opening time and mass dispensed in 100 cycles for all fluids is almost linear with R²=1. The curves shown in FIG. 3 show the Coefficient of Variance (CV) for different fluids. In all cases the CV stays below 2% and as the size of drop increases as Tau increases, it reduces to below 0.8%.

### Mechanical Dispensing Design.

The dispensing system consisted of a pressurized fluid reservoir (Ultra™ Dispensing system, EFD) which was connected to a pressure-regulated gas source using a barrel adapter assembly and to a VHS microdispensing unit (The Lee Co., CT). A Spike and Hold Driver (ICEX0501350A, The Lee Co., CT) provided a safe operating voltage profile for the Lee VHS valves by converting a TTL control signal into a spike and hold voltage which could be used by the VHS valves. The Lee VHS valve requires a voltage spike in order to actuate. The initial voltage spike is too high to allow continuous operation of the valve and therefore must be reduced immediately after the valve has been actuated. If the voltage is not reduced, the valve will overheat and experience permanent damage. The valve thus must be supplied with a control signal (5 vdc TTL), a hold voltage supply (3.5 vdc) and spike voltage supply (24 vdc). Voltage was supplied in the current setup by S82k-03024 and S82k-00705 power supplies (OMRON). For the TTL signal a PCI 6251 card (National Instruments) connected to a CB-68LP (National Instruments) board was used. The valve is operated using a LabView controlled computer interface. The gas inside the reservoir pushes the solution out through the dispensing valve when the latter is in an open position. There are three ways the dispensed volume can be changed:
The MINSTAC dispensing nozzle can be changed. Using larger or smaller orifice sizes will increase or decrease the dispensed volume respectively.
The inlet pressure can be changed. The inlet pressure directly affects the volume dispensed. If the pressure is increased, the dispensed volume increases proportionately. A typical starting pressure is 5 psi (at the valve)
The on-time of the valve can be changed.

All of these variables can be adjusted to find the best dispensing point for a specific fluid.

Below the valve a weighting module (WXSS205DU, Mettler Toledo, Ohio) interfaced with PC for automated weight recording. An in line camera was focused on the system which is also automatically controlled through Labview. The weighting module and dispensing system was placed inside an acrylic box to prevent air current from altering the process performance.

The fluid ejected by the valve was collected into gelatin capsules mimicking the operation of the capsule filling in a small dispensary or lab. The capsule was placed into a plastic holder that was mounted on the top of a weighting module. As the fluid was ejected based on user defined settings of pressure and valve opening time, the weight was measured in real time.

### Prep. of HPC Film.

HPC was dissolved into water at a material to water ratio of 1:19 VWR glass slides were cleaned with chromege solution and sulfuric acid. The slides were then rinsed with ethanol and allowed to dry. A solution of 5% HPC solution was cast onto the slides and placed in a vacuum oven at a temp of 30 degrees Celsius for a period of 4 hours for drying.

### Software Design.

The measuring process was fully automated by a data acquisition and experiment control program. The control program was developed using Labview (G-Language). The program is divided into two parts: pulse generation and weight measurement.

### Pulse Generation.

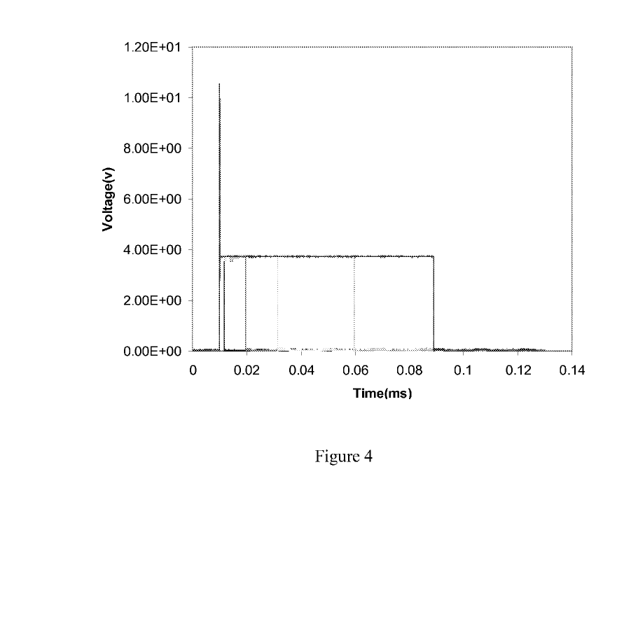

FIG. 4 shows the flow of the program used to create the pulses. The objective is to provide a control signal to the valve for actuation. The valve needs to be opened and then closed within a set period of time. The user defines the pulse duration and number of droplets. When the software is initialized, it opens a loop which encloses all parts of a program that opens and closes the valve a specified number of times with a delay. The structure is divided into two parts. The first generates a digital pulse, the second attaches an on/off signal to this pulse and sends it to the valve.

The Create Channel block in FIG. 4 is used to address a specific line on the port. It creates a virtual channel or a set of virtual channels and adds them to a task. The instances of this block correspond to the I/O type of the channel, such as analog input, digital output, or counter output. In this instance the block creates a channel to generate digital pulses that frequency defines. Because the valve opening and closing needs to be very accurate, a hardware clock is used to time these pulses and its address is supplied as input to the block. Thus the digital output is supplied to this block.

Next block is the DAQmx Start. It transitions the task to the running state to begin the generation. This block, though optional, is required because data is written to the channel multiple times in a loop. Without the presence of a start block the task starts and stops repeatedly which reduces the performance of the application.

The next block within a Write Data function which writes samples to the task or virtual channels specified. The data written is a Boolean one or zero depending on whether the digital pulse is high or low. (Boolean ‘one’ opens the valve and ‘zero’ closes it). A Wait block is added to introduce delay between drops, which waits the specified number of milliseconds and continues execution of the program. Finally, the program comes back into the main loop and checks whether it has executed the set number of times and if it has, the task is stopped by a Stop Task block.

### Driving Waveform.

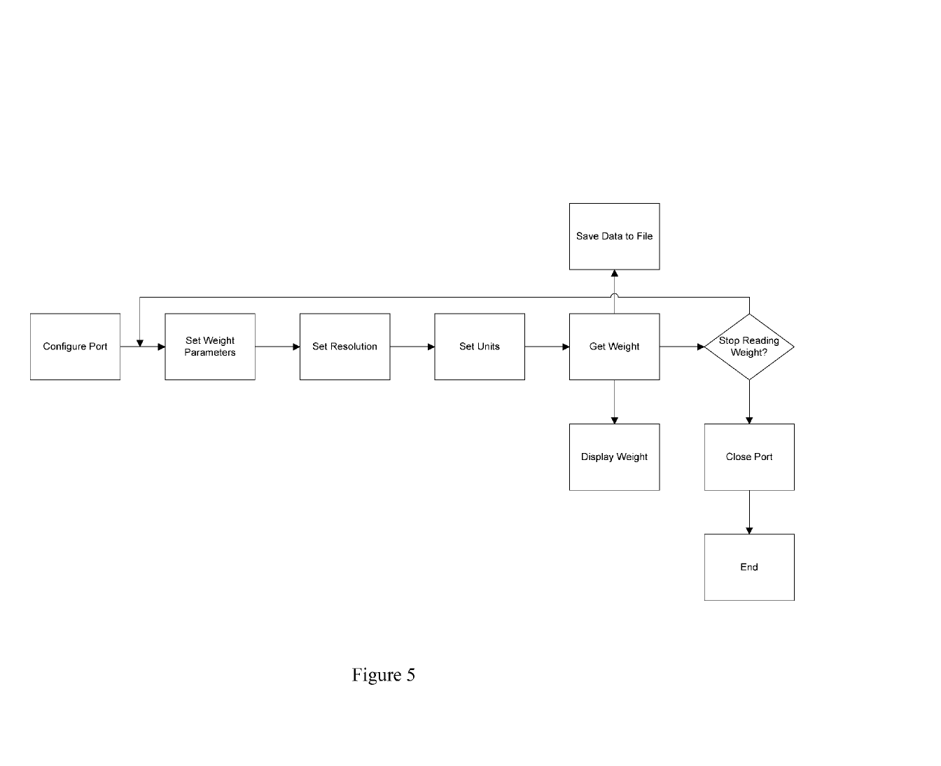

The control waveform, shown in FIG. 5, is provided by the Spike and Hold Driver (the Lee company) which assists in producing precision fluid dispense volumes from valves. Its use ensures optimized fluid dispensing while reducing the risk of overheating the valves. The module coverts a TTL control signal into the required waveform. The driver is pre-tuned to apply precise sculpted power pulses to the valve. The valve responds to the input waveform in the following manner. The valve requires a spike of power to actuate (this also reduces the response time). So initially a high voltage is supplied to it. The length of this spike is fixed. However this power level will generate more heat than the valve can safely dissipate. So after the valve has opened, the voltage must be reduced to prevent permanent damage to the valve. The voltage is reduced to lower value termed the hold voltage. This is required to keep the valve open. Once the TTL control signal ends the hold voltage is removed and the valve closes.

### Weight Measurement.

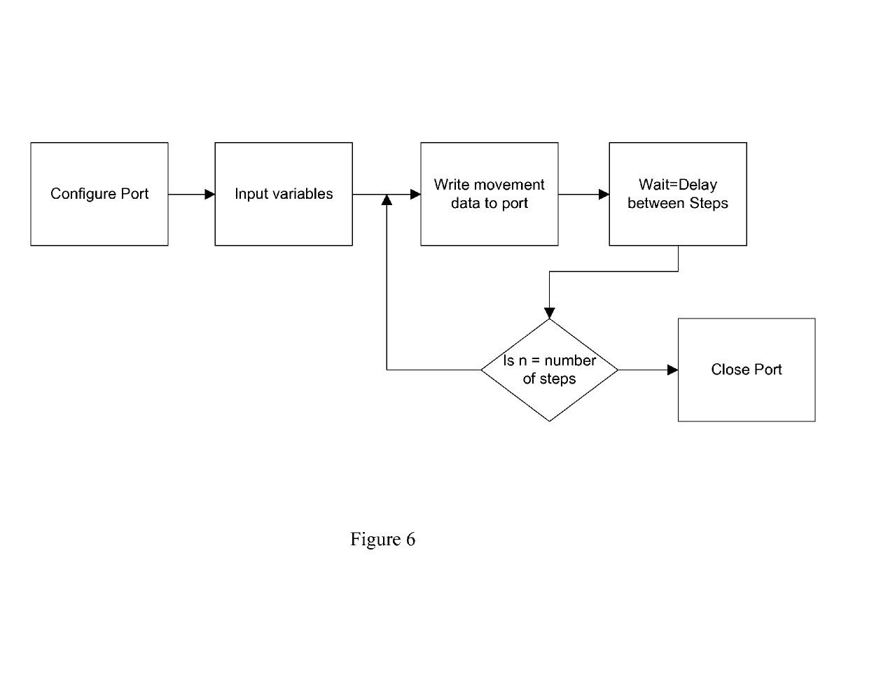

The second part of the program, shown in FIG. 6, is measures the weight of the samples. It initializes the balance, configures measurement and continuously takes dynamic weight measurement every 250 ms until user presses the stop button. The actual weight is displayed with an indicator and a chart graph. The user defined input parameters are Environment, Measurement Release and auto zero. Measurement release specifies how fast the balance will consider the measured value as stable. There are five levels ranging from very fast to very reliable. The repeatability of the measurement is lower with higher speed of measurement. Environment specifies the stability of the surroundings with respect to temperature fluctuations and vibration. This is again definable from very stable to very unstable. Auto Zero zeroes the scale before every measurement.

The Weighing module communicates through a RS 232 serial port, so in the first step the serial port is called using a LabVIEW command block. Once the serial port has been called, it is configured to function with the weighing module at the following settings: 9600 baud rate, eight data bits, Xon Xoff hardware handshake, one stop bit and no parity. Theses are the default values for the system. Once configured the port is opened for communication using the VISA architecture Open command. Next, a sequence of commands pertaining to the measurement setup, as defined by the user, are sent to the module using the VISA write command. Finally the measured weight is read in as an array and stored in a worksheet named by the user.

### Motorized Stage Motion Control.

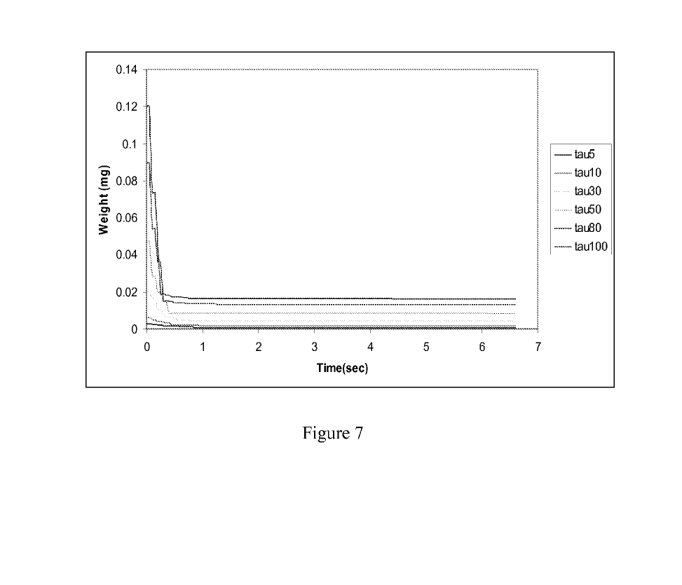

The motorized stage is operated through the serial port. Two sub VI (Virtual Instrument) routines are used to configure the port and move the stage. These can be incorporated into the Labview program for drop on demand system. The flow chart for this portion of the software is shown in FIG. 7.

The stage communicates through a RS 232 serial port, so in the first step the serial port is called using a Labview command block. For configuring the port, there are two user defined variables, which are the baud rate (based on manufacturer specifications) and the address of the com port. There are also predefined parameters with the following settings: eight data bits, no hardware handshake, ten stop bits and no parity. Theses are the default values for the system. Once configured, the VI for stage movement is called. In this VI the variables are: number of steps, movement on x axis, movement on y axis and delay between each step. A user defined time delay is also present between each movement. The displacement of the stage in x and y directions is determined by the user and sent to the port using the VISA write command. This sequence repeats movement in the same direction depending on the variable for number of steps. The VI can be used to make basic movements in x and y directions. The displacement is relative to the previous position of the stage.

### Discrete Operation Regime.

The addition of a single droplet is a dynamic process which depends on both the ambient pressure of the dispensed fluid as well as the length of time the valve is open. As the drop leaves the valve it hits the container surface and transfers the momentum causing dynamic perturbation of the WM output signal. This momentum force is given by:

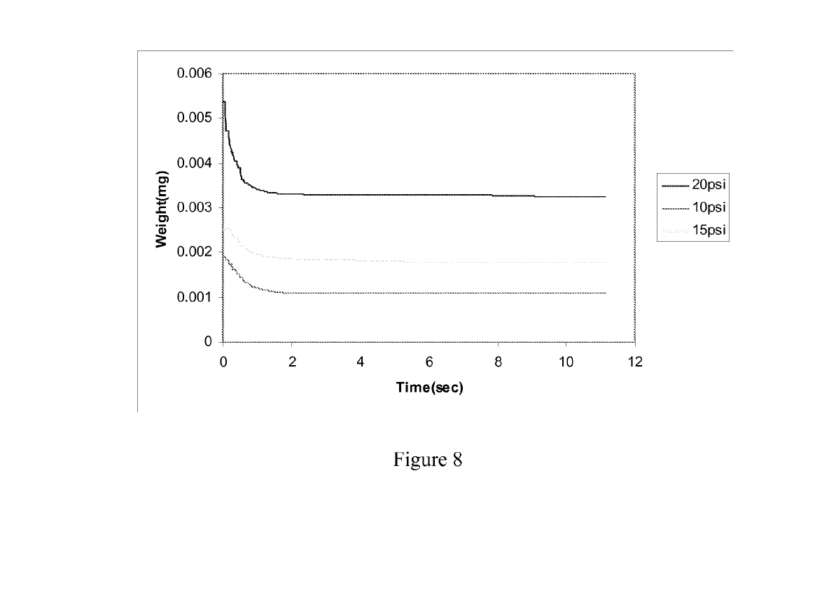

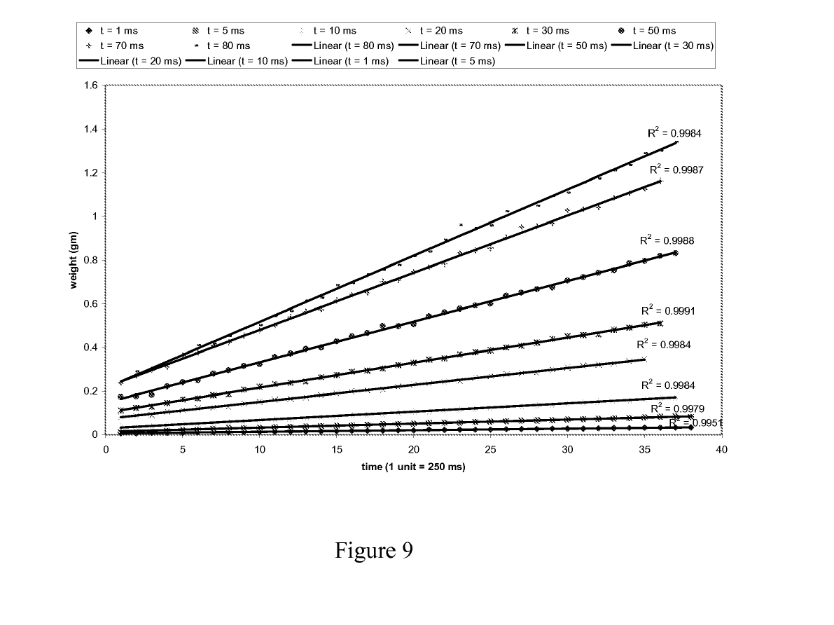

F˜Q\sqrt{ρΔP}

where F is the force, ΔP is the pressure difference inside and outside the valve. The effect of this force can be seen by the response of the weighing module as depicted in FIGS. 8 and 9. First the measured weight increases and then it settles down to a steady state which is the true weight of the droplet. For dosage determination of true weight is critical as it will be used as a control parameter. The weight is considered true when Del W/W<0.01. This condition occurs only after 1 second from first measurement.

### Continuous Operation Regime.

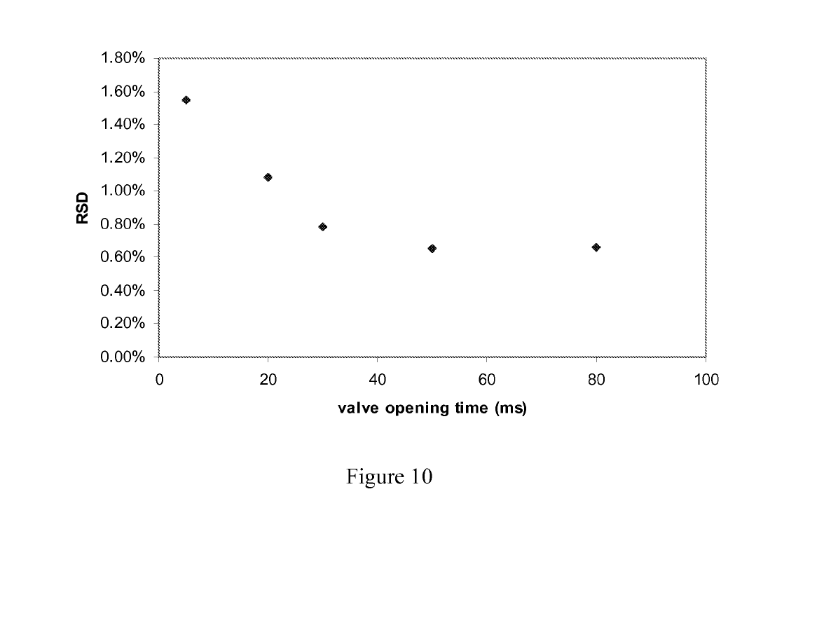

The precision of the dispensing system is tested by recording the mass of aliquots as they are collected on a plastic tray. FIG. 10 plots the mass of DI water on a tray versus time (readings collected every 250 ms). The different curves in FIG. 10 correspond to different times used to keep the valve opened. The longer the opening period for the valve, the larger the mass of water dispensed.

Testing the System with Different Solutions.

Finally, the micro-dispensing system is used to administer model drugs, with a solution of drug, solvent and polymer that are relevant compositions for many pharmaceutical processes. The linear correlation between the opening time for the valve and the mass of solution dispensed after 100 cycles can be seen in FIG. 5. However, different amounts of the different fluids pass through the valve while it is open for a specific period of time. A correlation can be seen between the viscosity and the mass of fluid dispensed. The mass of liquid dispensed increases from ‘CP with PEG,’ a comparably viscous solution, to ethanol which is least viscous. However, the correlation between valve opening time and mass dispensed in 100 cycles for all fluids is almost linear with R²=1. The curves in FIG. 2 serve as calibration curves for the system for each different fluid.

### Filling Mode.

The first aspect assessed is the reproducibility of aliquots. For a given opening time of the valve, every fluid portion dispensed into the receiving container should increase the total mass by a constant value and the WM reading should increase linearly. Therefore, the aliquot reproducibility is assessed by measuring the deviation of the balance recording (total fluid mass versus the number of aliquots dispensed) from a straight line. The results show that, in general, the gravimetric readings fit a straight line with an R²≧0.995.

Another aspect to be assessed is the ability of the system to dispense any desired amount of model fluid with the appropriate combination of cycles and opening time of the valve. The opening time of the valve is a critical parameter in the design of a dosage expending protocol. For example, using the system for 100 cycles and different opening times of the valve, a correlation between the latter and the fluid mass dispensed can be established. Our data shows that the mass of water dispensed after 100 cycles increases linearly with the time used for the opening of the valve. The correlation between VOT and the fluid mass dispensed in 100 cycles is perfectly linear with R²=1. Therefore, it is possible to design microdispensing operation with the high level of accuracy and reproducibility.

Lastly, the capability of the system to administer dosages with high repeatability is tested. In order to accomplish this aim, each experimental condition (i.e. 100 cycles with a specific time of opening for the valve) is tested 10 times. The standard deviation, s, of the 10 dosages is estimated as well as the relative standard deviation (RSD). These terms are defined as:

$$R S D = \frac{s}{W} \\ s = \sqrt{\frac{n {\sum }_{i = 1}^{n} {w}_{i}^{2} - {( {\sum }_{i = n}^{n} {w}_{i} )}^{2}}{n ( n - 1 )}} \qquad ( 1 )$$

where s is the standard deviation of all sample concentrations, W is the average weight of 100 aliquots, wᵢ is the mass of each individual aliquot and n is the total number of experiments with 100 aliquots (10 experiments).

Experimental data indicates that the standard deviation of dosage mass increases linearly with the time used in the protocol for the opening of the valve. FIG. 11 shows that the RSD first decreases and for larger opening times stays at a constant value. For the operating system and the hardware used in these experiments, RSD is generally better than 1%.

FIG. 11 can be used as a calibration curve for the dispensing system and to establish the dosage error for a protocol with a specific time of opening for the valve respectively. In order to minimize the RSD of larger doses beyond 0.6% while still keeping a high throughput, it may be possible to use a protocol that combines large and small aliquots. However, the optimum combination of aliquots for a specific dosage is a matter of an additional experimental study.

### Control Algorithm and Continuous System Operation.

In the current measurement method, only the droplet volume/mass is deduced, and the full system information, such as the exact API content in individual droplet, is missing. A mathematical model cannot fully depict the uncertainties of surface condition and device variation in any case. Through experimental study, basic control algorithms can be explored, by measuring the droplet volume dynamic responses but without making a detailed analysis, such as its stability, robustness, or system error control.

To characterize the droplet volume change under the feedback control, the dynamic response of weighting module was recorded. As shown in FIG. 5, changing a valve opening time caused the droplet volume to rapidly decrease into the controlled range. The real-time feedback control should converge as fast as possible to maintain a stable volume. Droplet volume can be regulated via several process parameters: supplied gas pressure, nozzle diameter and time of valve opening. It is clear that the time of single droplet dispensing is the easiest parameter to control electronically in real-time. A quite sophisticated feedback controller can be designed with the time of valve opening as control output and measurement mass of deposited droplet as control target. Since proportional control alone is not enough, integral and differential components may be added into the feedback control algorithm to improve the overall accuracy of DoD operations. The discrete time proportional-integral-derivative (PID) feedback controller for the droplet volume control is:

τₙ₊₁=Kᵢτₙ+Kₚεₙ+K_{d}(εₙ−εₙ₋₁) (2)

where, τₙ is the time of valve opening for n-th droplet, εₙ is the error εₙ=mₙ−m₀ with mₙ, m₀—mass of generated n-th droplet and target droplet mass respectively. Kₚ and K_{d} are proportional and differential coefficients, respectively. The integral coefficient Kᵢ is always kept at one to ensure that the discrete time feedback control system is stable. Different values of Kₚ and K_{d} can be explored experimentally accompanied with measured droplet volume response. The overall operation of the DoD system can be significantly improved under the PID control. Based on the general principle of PID control, with large K_{d} and smaller Kₚ, the system tends to converge faster but may exhibit larger deviation from the target value. By properly selecting K_{d} and Kₚ, the feedback control can converge fast enough while keeping the system error small.

If dispensed droplet volume in is less than the target volume, the next droplet should be generated with valve opened for the longer period of time. When the feed-back control is stable and converges fast enough, less than 1% droplet volume precision can be expected by real-time feedback control.

### List of Companies

There are a few companies which are producing systems which incorporate individually or a combination of the technologies discussed herein.

Innovadyne's (Santa Rosa, Calif.) Nano Drop™ system has a hybrid syringe-microsolenoid valve technology that can dispense as low as 50 nl of 35% PEG 8000.

Labcyte's (Sunnyvale, Calif.) Echo 550 performs direct microplate-to-microplate transfers of droplets down to 2.5 mL. EDC Biosystems offers the ATS-100 acoustic transfer system, which can transfer volumes from 1-250 mL with coefficients of variation (CV) lower than 10%.

The Lee Company offers the VHS micro dispensing solenoid valve having a broad range of chemical compatibility and a very fast, stable response time providing repeatable dispenses in the 100 nanoliter to 500 microliter range.

MARKEM Corp. (Keene, N.H.), New Systems (Italy), and others already market equipment designed to print component legends and other labeling information onto printed circuit boards.4\ Companies such as Printar (Israel), New Systems, and Patterning Technologies Ltd. (U.K.) are commercializing printers that extend inkjet print heads to the printing of etch resists, solder masks, solder, and conductive traces. Avecia (U.K.) and Cabot Superior MicroPowders (Albuquerque, N.M.) market jettable fluids for these applications

Microfab Technologies produces the Jetlab™ table top printing platform based on piezoelectric transducer that has found use in a range of applications.

| | | | | |
|---|---|---|---|---|
| Piezoelectric | Solenoid | Thermal | Acoustic | Positive |
| Microfab | Lee | Olivetti | Labcyte | Displacement |
| Dimatix Fuji Films | Company | I-Jet | | New Era Pump |
| Epson | Innovadyne | Canon | | Systems, Inc |
| Samsung Electro- | | HP | | I&F Fisnar |
| Mechanic, Inc | | | | Kent Scientific |
| | | | | Corporation |

Comparative analysis of products from different companies

| | MICROFAB | | THE LEE | | | | I&S | |
|---|---|---|---|---|---|---|---|---|
| | TECHNOLOGIES, | | COMPANY (VHS | | | | FISNAR | |
| Attributes | INC.(MJ-SF-01) | | Dispensing valve) | | EFD (Picodot) | | (VDP150) | |
| Viscosity of | less than 40 cP | | less than 40 cP | | 50-500,000 | cP | Up to 1000 cP | |
| fluid | | | | | | | | |
| Operating | 20° C. to 150° C. | | 4° C. to 71° C. | | up to 100° C. | | Not suited for | |
| Temp | | | | | | | heating | |
| Speed (Jetting | 30 | Khz | 1.2 | khz | 1 | Khz | 100 | Hz |
| Frequency) | | | | | | | | |
| Life Cycle | 25-40 | billion | 500 | million | 1 | billion | 1 | billion |
| Metering | ~2% | | ~1% | | ~2% | | ~1% | |
| Accuracy (CV) | | | | | | | | |
| Drop Size | 50-200 | picoliter | From 10 nl | | From 2 nl | | From 5 | |
| | | | | | | | microliter | |
| Cost (complete | ~16000 | USD | ~500 | USD | ~21000 | USD | ~1000 | USD |
| system) | | | | | | | | |

### Working Mechanisms

MicroFab Technologies, Inc. (MJ-SF-01)—The MJ-SF device consists of an annular piezoelectric actuator bonded to a glass capillary that is connected at one end to the fluid supply and at the other end has an orifice generally in the range of 30 to 60 um. By applying a voltage to the PZT actuator, the cross-section of the tube capillary is reduced/increased producing pressure variations of the fluid enclosed in the tube. These pressure variations propagate in the glass tube towards the orifice. The sudden change in cross-section (acoustic impedance) at the orifice, causes a drop to be formed. A wide range of fluids can be dispensed with the requirement that the viscosity has to be lower than 40 centipoise. Drop volume is a function of the fluid, orifice diameter, and actuator driving parameters (voltage and timings) usually ranging from 50 picoliters to 200 picoliters. The operating frequency is limited by the total driving time of the actuator and on the dispensed fluid.

THE LEE COMPANY (VHS Dispensing valve)—The dispensing system consists of a pressurized fluid reservoir (Ultra™ Dispensing system, EFD), which is connected to a pressure-regulated gas source using a barrel adapter assembly and to the VHS microdispensing unit (Lee Co.) The spike and hold driver (ICEX0501350A, Lee) provides a safe operating voltage profile for the Lee VHS valves by converting a TTL control signal into a spike and hold voltage that can be used by the VHS valves. The Lee VHS valve requires a voltage spike in order to actuate. The initial voltage spike is too high to allow continuous operation of the valve and must be reduced immediately after the valve has been actuated. If voltage is not reduced, the valve will overheat and experience permanent damage. The valve has to be supplied a control signal (5 vdc TTL), hold voltage supply (3.5 vdc) and a spike voltage supply (24 vdc). Voltage is supplied in the current setup by S82k-03024 and S82k-00705 power supplies (OMRON). For the TTL signal a PCI 6251 card (National Instruments) connected to a CB-68LP (National Instruments) board is used. The valve is operated using a LabView controlled computer interface. The gas inside the reservoir pushes the solution out through the dispensing valve when the latter is in an open position.

I&S FISNAR (VDP150)—The VDP150 positive displacement valve was developed for dispensing small shots of low and medium viscosity materials. These valves are powered by timed air pulses that open seals or gates which let a material flow. Return springs close the seals. The valve operates by the movement of the plunger. When the plunger goes down, the material sucked into the valve chamber is dispensed. On the other hand, when plunger goes up, material is sucked into the valve chamber because of the negative pressure.

It will be realized by one of skill in the art that many different mechanical embodiments are possible and that the exemplary equipment and system described herein is only one possible embodiment. Likewise, one of skill in the art will realize that the control software may be implemented in many different ways and using many different languages. The exemplary flow charts representing the control software presented herein are only one possible embodiment. All mechanical embodiments as well as all possible control software implementations are contemplated to be within the scope of the invention.

1. A system for the manufacture of a custom dose of one or more pharmaceutically active agents comprising:
a. a metering system, to provide for the precise deposition of micro-quantities of one or more pharmaceutically active agents;
b. a chemical analyzer, to non-destructively verify the composition and structure of said deposition; and
c. a microprocessor interfaced to said metering system and said chemical analyzer to control the deposition and verification of said pharmaceutically active agent.

2. The system of claim 1 characterized in that it is configured to deposit more than one pharmaceutically active agent into a single pharmaceutically acceptable medium.

3. The system of claim 2 characterized in that it is configured to deposit a different concentration of each of the pharmaceutically active agents.

4. The system of claim 1 characterized in that it is configured to manufacture a plurality of different custom doses.

5. The system of claim 1 characterized in that it is configured to manufacture a plurality of the same custom doses.

6. The system of claim 1 characterized in that it is configured to also provide one or more pharmaceutically acceptable excipients.

7. The system of claim 6 wherein said one or more pharmaceutically acceptable excipients are selected from the group consisting of surfactants, preservatives, stabilizers, biocompatible polymers, solvents, viscosity modifiers, absorption enhancers, mucoadhesives, solvents, buffers, acidulants, diluents, emulsifying agents, suspending agents, wetting agents, anti-caking agents, plasticizers, coating agents, sweetening agents, flavor enhancers, flavoring agents, coloring agents, adsorbents and antioxidants.

8. The system of claim 1 further comprising a mechanical handling system to handle a pharmaceutically acceptable medium into which said pharmaceutically active agent is deposited.

9. The system of claim 8 wherein said medium is selected from the group consisting of non-consumable mediums, consumable mediums, capsules, syringes, and vials.

10.-11. (canceled)

12. The system of claim 1 wherein said pharmaceutically active agent is selected from the group consisting of chemotherapeutic agents, agents for treating central nervous system disorders, agents for treating allergic reactions, agents for treating attention deficit disorder, micronutrients, vitamins, agents for treating human immunodeficiency virus, hormone therapy agents, anticoagulants, highly potent bio-pharmaceuticals, agents for treating pediatric disorders, agents for treating geriatric disorders, diagnostic agents, radiopharmaceutical agents, gastrointestinal drugs, liver drugs, blood, fluids, electrolytes, hematological drugs, cardiovascular drugs, respiratory drugs, sympathomimetic drugs, cholinomimetic drugs, adrenergic antagonists, adrenergic neuron blocking drugs, antimuscarinic drugs, antispasmodic drugs, skeletal muscle relaxants, diuretic drugs, uterine drugs, anti-migraine drugs, hormones, hormone antagonists, general anesthetics, local anesthetics, anti-anxiety drugs, hypnotic drugs, antiepileptic drugs, psychopharmacologic drugs, analgesics, antipyretics, anti-inflammatory drugs, histamine, anti-histaminic drugs, central nervous system stimulants, anti-neoplastic drugs, immunoactive drugs, parasiticides, immunizing agents, allergenic extracts, anti-infectives, enzymes, nutrients, vitamins, micronutrients, nutraceuticals and pesticides.

13. The system of claim 1 wherein said microprocessor comprises memory programmed with a database characterized by information on various pharmaceutical preparations and with instructions for controlling said metering system and said chemical analyzer to create said various pharmaceutical preparations.

14. The system of claim 1 further comprising a reading or communication device for receiving a dosage formulation from a paper or electronic prescription.

15. The system of claim 1 wherein said dose is selected from the group consisting of geriatric doses and pediatric doses.

16. (canceled)

17. A method for preparing a custom dose comprising at least one pharmaceutically active agent utilizing the system of claim 1, comprising providing instructions to said microprocessor selecting the custom dose to be prepared and commanding the micro-processor to operate said system.

18. The method of claim 17 wherein said custom dose comprises more than one pharmaceutically active agent in a single pharmaceutically acceptable medium.

19. The method of claim 18 wherein said custom dose comprises different concentrations of each of the pharmaceutically active agents.

20. The method of claim 17 wherein said instructions select a plurality of different custom doses.

21. The method of claim 17 wherein said instructions select a plurality of the same custom doses.

22. A custom dose of at least one pharmaceutically active agent prepared according to the method of claim 17.

23. The dose of claim 22 comprising more than one pharmaceutically active agent in a single pharmaceutically acceptable medium.

24. The dose of claim 23 comprising different concentrations of each of the pharmaceutically active agents.

25. A plurality of the doses of claim 22 characterized by a plurality of different custom doses.

26. A plurality of the doses of claim 22 characterized by a plurality of the same custom doses.

27. A dose according to claim 22 characterized by being formulated for a geriatric patient or a pediatric patient.

28. (canceled)

29. A dose according to claim 22 comprising a pharmaceutically acceptable medium selected from the group consisting of consumable mediums, non-consumable mediums, capsules, syringes and vials.

30. (canceled)

31. A dose according to claim 22 wherein said pharmaceutically active agent is selected from the group consisting of chemotherapeutic agents, agents for treating central nervous system disorders, agents for treating allergic reactions, agents for treating attention deficit disorder, agents for treating human immunodeficiency virus, hormone therapy agents, anticoagulants, highly potent biopharma-ceuticals, agents for treating pediatric disorders, agents for treating geriatric disorders, diagnostic agents, radiopharmaceutical agents, gastrointestinal drugs, liver drugs, blood, fluids, electrolytes, hematological drugs, cardiovascular drugs, respiratory drugs, sympathomimetic drugs, cholinomimetic drugs, adrenergic antagonists, adrenergic neuron blocking drugs, antimuscarinic drugs, antispasmodic drugs, skeletal muscle relaxants, diuretic drugs, uterine drugs, anti-migraine drugs, hormones, hormone antagonists, general anesthetics, local anesthetics, anti-anxiety drugs, hypnotic drugs, antiepileptic drugs, psychopharmacologic drugs, analgesics, antipyretics, antiinflammatory drugs, histamine, anti-histaminic drugs, central nervous system stimulants, anti-neoplastic drugs, immunoactive drugs, parasiticides, immunizing agents, allergenic extracts, anti-infectives, enzymes, nutrients, vitamins, micronutrients, nutraceuticals and pesticides.

32. (canceled)

33. The method of claim 17 wherein said dose is prepared prior to dispensing to a patient or to a participant in a clinical trial.

34. (canceled)

35. The method of claim 33 wherein said dose is formulated for a pediatric patient or a geriatric patient.

36. (canceled)

37. The method of claim 33 wherein said patient is a pediatric patient or a pediatric patient.

38.-39. (canceled)

40. The method of claim 17 wherein said dose is prepared in the course of pharmaceutical research.

41. The method of claim 17 wherein said dose is prepared to order from an order or prescription issued by a professional with authority to prescribe or order the dispensing of drugs.
